(19) United States
(12) Patent Application Publication
(10) Pub. No.: US 2022/0148731 A1
(43) Pub. Date: May 12, 2022

(54) Systems and Methods for Uncertainty Quantification in Radiogenomics

(71) Applicant: Mayo Foundation for Medical Education and Research, Rochester, MN (US)

(72) Inventors: Andrea J. Hawkins-Daarud, Houston, TX (US); Leland S. Hu, Phoenix, AZ (US); Kristin R. Swanson, Phoenix, AZ (US); Teresa Wu, Gilbert, AZ (US); Jing Li, Marietta, GA (US); Lujia Wang, Scottsdale, AZ (US)

(21) Appl. No.: 17/524,591

(22) Filed: Nov. 11, 2021

Related U.S. Application Data
(60) Provisional application No. 63/112,496, filed on Nov. 11, 2020.

(51) Int. Cl. G16H 50/20; G16H 30/40; G06N 5/02; A61B 5/055; A61B 5/026; A61B 5/00; G16B 5/20; G16B 40/00

(52) U.S. Cl. CPC G16H 50/20; G16H 30/40; G06N 5/022; A61B 5/055; A61B 2576/026; A61B 5/0042; A61B 5/7267; G16B 5/20; G16B 40/00; A61B 5/0263

(57) ABSTRACT

Genetic and/or other biological marker prediction data are generated based on inputting medical image data to a suitably trained machine learning model, where the output genetic prediction data not only indicate a prediction of genetic features and/or other biological markers for a subject, but also a measure of uncertainty in each of those predictions. As an example, a transductive learning Gaussian process model is used to generate the genetic and/or other biological marker predication data and corresponding predictive uncertainty data. As another example, a knowledge-infused global-local data fusion model can be used for spatial predictive modeling.

### CROSS-REFERENCE TO RELATED APPLICATIONS

This application claims the benefit of U.S. Provisional Patent Application Ser. No. 63/112,496, filed on Nov. 11, 2020, and entitled “SYSTEMS AND METHODS FOR UNCERTAINTY QUANTIFICATION IN RADIOGENOMICS,” which is herein incorporated by reference in its entirety.

### STATEMENT REGARDING FEDERALLY SPONSORED RESEARCH

This invention was made with government support under NS082609, CA221938, and CA220378 awarded by the National Institutes of Health, and under NSF1149602 awarded by the National Science Foundation. The government has certain rights in the invention.

### BACKGROUND

The field of machine-learning (ML) has exploded in recent years, thanks to advances in computing power and the increasing availability of digital data. Some of the most exciting developments in ML have centered on computer vision and image recognition, with broad applications ranging from e-commerce to self-driving cars. But these advances have also naturally dovetailed with applications in healthcare, and in particular, the study of medical images. The ability for computer algorithms to discriminate subtle imaging patterns has led to myriad ML tools aimed at improving diagnostic accuracy and clinical efficiency. But arguably, the most transformative application relates to the development of radiogenomics modeling, and its integration with the evolving paradigm of individualized oncology.

Radiogenomics integrates non-invasive imaging (typically Magnetic Resonance Imaging, or MRI) with genetic profiles as data inputs to train ML algorithms. These algorithms identify the correlations within the training data to predict genetic aberrations on new unseen cases, using the MRI images alone. In the context of individualized oncology, radiogenomics non-invasively diagnoses the unique genetic drug sensitivities for each patient's tumor, which can inform personalized targeted treatment decisions that potentially improve clinical outcome. Foundational work in radiogenomics coincided with the first Cancer Genome Atlas (TCCA) initiative over a decade ago, which focused on patients with Glioblastoma (GBM)—the most common and aggressive primary brain tumor. Since then, radiogenomics has extended to all major tumor types throughout the body, underscoring the broad scope and potential impact on cancer care in general.

But as clinicians begin to assimilate radiogenomics predictions into clinical decision-making, it will become increasingly important to consider the uncertainty associated with each of those predictions. In the context of radiogenomics, predictive uncertainty stems largely from sparsity of training data, which is true of all clinical models that rely on typically limited sources of patient data. This sparsity becomes an even greater challenge when evaluating heterogeneous tumors like GBM, which necessitate image-localized biopsies and spatially matched MRI measurements to resolve the regional genetic subpopulations that comprise each tumor. And as with any data driven approach, the scope of training data establishes the upper and lower bounds of the model domain, which guides the predictions for all new unseen test cases (i.e., new prospective patients). In the ideal scenario, the new test data will fall within the distribution of the training domain, which allows for interpolation of model predictions, and the lowest degree of predictive uncertainty. If the test data fall outside of the training domain, then the model must extrapolate predictions, at the cost of greater model uncertainty. Unfortunately, without knowledge of this uncertainty, it is difficult to ascertain the degree to which the new prospective patient case falls within the training scope of the model. This could limit user confidence in potentially valuable model outputs.

While predictive accuracy remains the most important measure of model performance, many non-medical ML-based applications (e.g., weather forecasting, hydrologic forecasting) also estimate predictive uncertainty—usually through probabilistic approaches—to enhance the credibility of model outputs and to facilitate subsequent decision-making.9 In this respect, research in radiogenomics has lagged. Past studies have focused on accuracy (e.g., sensitivity/specificity) and covariance (e.g., standard error, 95% confidence intervals) in group analyses, but have not yet addressed the uncertainty for individual predictions.10 Again, such individual predictions (e.g. a new prospective patient) represent the “real world” scenarios for applying previously trained radiogenomics models. Quantifying these uncertainties will help to understand the conditions of optimal model performance, which will in turn help define how to effectively integrate radiogenomics models into effective clinical practice.

### SUMMARY OF THE DISCLOSURE

The present disclosure addresses the aforementioned drawbacks by providing a method for generating genetic prediction data, or other biological prediction data from medical image data. A trained machine learning model is accessed with a computer system, wherein the machine learning model has been trained in order to generate genetic prediction data, or other biological prediction data and corresponding predictive uncertainty data from medical image data. The machine learning model may be a Gaussian process model, which may be trained using transductive learning. Medical image data are accessed with the computer system, wherein the medical image data comprise medical images of a subject obtained using a medical imaging system. The medical image data are input to the trained machine learning model, generating output as genetic prediction data, or other biological prediction data and corresponding predictive uncertainty data, wherein the genetic prediction data, or other biological prediction data comprise genetic predictions and corresponding predictive uncertainty data comprise quantitative measures of an uncertainty of each prediction in the genetic prediction data, or other biological prediction data. The genetic prediction data, or other biological prediction data and predictive uncertainty data can then be displayed to a user.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a mathematical formulation of a knowledge-infused global-local data fusion model as a constrained optimization.

FIG. 2 is a flowchart setting forth the steps of an example method for training a machine learning model, such as a Gaussian process model or a knowledge-infused global-local data fusion model, to generate output as genetic prediction data, or other biological prediction data and corresponding predictive uncertainty data.

FIG. 3 is a flowchart setting forth the steps of an example method for generating genetic prediction data, or other biological prediction data and corresponding predictive uncertainty data by inputting medical image data to a suitably trained machine learning model.

FIG. 4 illustrates how radiogenomics maps resolve the regional intratumoral heterogeneity of EGFR amplification status in GBM. Shown are two different image-localized biopsies (Biopsy #1, Biopsy #2) from the same GBM tumor in a single patient. For each biopsy, T1+C images (left) demonstrate the enhancing tumor segment (dark green outline, T1W+Contrast) and the peripheral non-enhancing tumor segment (light green outline, T2W lesion). Radiogenomics color maps for each biopsy (right) also show regions of predicted EGFR amplification (amp, red) and EGFR wildtype (wt, blue) status overlaid on the T1+C images. For biopsy #1 (green square), the radiogenomics map correctly predicted low EGFR copy number variant (CNV) and wildtype status with high predictive certainty (p<0.5). Conversely for biopsy #2 (green circle), the maps correctly predicted high EGFR CNV and amplification status, also with high predictive certainty (p<0.5). Note that both biopsies originated from the non-enhancing tumor segment, suggesting the feasibility for quantifying EGFR drug target status for residual subpopulations that are typically left unresected followed gross total resection.

FIG. 5 illustrates how model performance increases with lower predictive uncertainty. Shown are the Area-under-the-curve (AUC) measures for Receiver Operator Characteristics (ROC) analysis of sample predictions stratified by predictive uncertainty. The sample predictions with lowest uncertainty (p<0.5) (blue curve, n=72) achieved the highest performance (AUC=0.86) compared to the entire grouped cohort irrespective of uncertainty (AUC=0.83) (black curve, n=95). Meanwhile, the sample predictions with greatest uncertainty (i.e., least certain predictions) showed the lowest classification accuracy (AUC=0.5) (red curve, n=23).

FIG. 6 illustrates how model uncertainty can inform the likelihood of achieving an accurate radiogenomics prediction for EGFR amplification status. Two separate biopsies (#1 and #2) were obtained from the same tumor in a 44 year-old male patient with primary GBM. The (A) T2W and (B) T1+C images demonstrate the enhancing (dark green outline, T1W+Contrast) and peripheral non-enhancing tumor segments (light green outline, T2W lesion). The (C) radiogenomics color map shows regions of predicted EGFR amplification (amp, red) and EGFR wildtype (wt, blue) status overlaid on the T1+C images. For biopsy #1 (green circle), the radiogenomics model predicted EGFR wildtype status (blue) with high certainty, which was concordant with biopsy results (green box). For biopsy #2 (yellow circle), the radiogenomics model showed poor certainty (i.e., high uncertainty), with resulting discordance between predicted (red) and actual EGFR status (yellow box).

FIG. 7 illustrates an example model training procedure for a knowledge-infused global-local data fusion model.

FIG. 8 shows example predicted tumor cell density (“TCD”) maps within an ROI for two patients in an example study, in which the TCD maps are shown as a color map overlaid on the respective patient's T2 MRI. Predicted variances are shown in distribution underneath each TCD map.

FIG. 9 is a block diagram of an example system for generating genetic prediction data, or other biological prediction data and corresponding predictive uncertainty data.

FIG. 10 is a block diagram of example components that can implement the system of FIG. 9.

### DETAILED DESCRIPTION

Described here are systems and methods for generating genetic prediction data, or other biological prediction data based on inputting medical image data to a suitably trained machine learning model and/or algorithm, where the output genetic prediction data, or other biological prediction data not only indicate a prediction of genetic or other biological features for a subject, but also a measure of uncertainty in each of those predictions. Current methods radiogenomics based prediction models are evaluated using an overall accuracy number (e.g., “this model is 89% accurate”), but do not provide a way to quantify the uncertainty of individual predictions (e.g., a quantitative measure of how certain the genetic and/or biological data obtained for this particular patient are trustworthy). In this way, the output genetic prediction data, or other biological prediction data can be used to clinically integrate reliable radiogenomics predictions as part of decision support within the paradigm of individualized oncology. Predictive uncertainty can inform the likelihood of achieving an accurate sample prediction, which enhances clinical interpretability.

In a non-limiting example, the systems and methods described in the present disclosure implement a transductive Gaussian process (“GP”), which is trained to minimize uncertainty rather than to maximize accuracy. As a result, individual genetic and/or other biological predictions output by the machine learning model and/or algorithm are output with a corresponding uncertainty figure. Collectively, the genetic and/or other biological marker prediction and predictive uncertainty can enable a clinician to assess the extent to which they can trust the results given by the model for a given patient.

Advantageously, the predictive uncertainty data output by the machine learning model and/or algorithm can also be used to enhance model performance and interpretability. Taking uncertainty into account in the training process allows for the uncertainty to be decreased, and also for the overall accuracy of the model to be increased compared to previous works.

In general, the machine learning models and/or algorithms based on a transductive GP method can quantify predictive uncertainty through probability distributions of regional genetic or other biological marker aberrations in each patient's tumor or other anatomical region-of-interest (“ROI”).

The era of genomic profiling has ushered new strategies for improving cancer care and patient outcomes through more personalized therapeutic approaches. In particular, the paradigm of individualized oncology can optimize targeted treatments to match the genetic aberrations and drug sensitivities for each tumor. This holds particular relevance for many aggressive tumors that may not be amenable to complete surgical resection, such as GBM or pancreatic adenocarcinoma, or other cancers that have widely metastasized. In these cases, the targeted drug therapies represent the primary lines of defense in combating the residual, unresected tumor populations that inevitably recur and lead to patient demise.

Yet, a fundamental challenge for individualized oncology lies in the inability to resolve the internal genetic heterogeneity of these tumors. Taking GBM as an example, each patient tumor actually comprises multiple genetically distinct subpopulations, such that the genetic drug targets from one biopsy location may not accurately reflect those from other parts of the same tumor. Tissue sampling errors are magnified by the fact that surgical targeting favors MRI enhancing tumor. With recent advances in the field of radiogenomics, image-based modeling offers a promising and clinically feasible solution to bridge the challenges of intratumoral heterogeneity. The past decade has seen a growing number of published studies comparing MRI with genetic profiles in GBM, typically in a non-localizing fashion.

Using machine-learning (ML) and image-localized biopsies, spatially resolved radiogenomics maps can quantify the regional intratumoral variations of key GBM driver genes, at the image voxel level, throughout different regions of the same tumor. This includes highly relevant drug targets such as Epidermal Growth Factor Receptor (EGFR), which amplifies in as many as 30-45% of GBM tumors, and serves as the target for many clinically tested and commercially available inhibitors. Importantly, these radiogenomics maps of intratumoral heterogeneity offer a clinically viable solution for diagnosing the potentially unique drug targets within the residual unresected tumor segment, which otherwise represent a major barrier to individualized oncology.

As the data available to train machine learning models is never fully representative of the predictive cases, model predictions always have some inherent uncertainty in their predictions. To translate these models into clinical practice, the radiogenomics predictions need to not only be accurate, but must also inform the clinician of the confidence or certainty of each individual prediction. This underscores the importance of quantifying predictive uncertainty, but also highlights the major gaps that currently exist in this field. To date, no published studies have addressed predictive uncertainty in the context of radiogenomics. In large part, this uncertainty stems from the inherent sparsity of training data, which limits the scope of the model domain. As such, there exists a certain degree of probability that any new unseen case may fall outside of the model domain, which would require the model to generalize or extrapolate beyond the domain to arrive at a prediction. All clinical models suffer from this sparsity of data, particularly when relying on spatially resolved biopsy specimens in the setting of intratumoral heterogeneity. But, knowledge of which predicted cases fall within or outside of the model domain will help clinicians make informed decisions on whether to rely upon or disregard these model predictions on an individualized patient-by-patient basis.

The transductive learning GP model described in the present disclosure addresses the challenges of predictive uncertainty at various levels. First, the model training incorporates the minimization of uncertainty as part of feature selection, which inherently optimizes the model domain to prioritize interpolation rather than extrapolation. It is contemplated that interpolated predictions generally correspond with higher predictive accuracy compared with extrapolated ones. Thus, by shifting the model domain towards interpolation, model performance rises.

Second, along these same lines, the GP model can inform the likelihood of achieving an accurate prediction by quantifying the level of uncertainty for each sample prediction. A dramatic increase in classification accuracy can be observed among those sample predictions with low uncertainty (p<0.5), which corresponded with interpolated predictions. This allows the clinician to accept the reality that not all model predictions are correct. But, by qualifying each sample prediction, the model can inform of which patient cases should be trusted over others.

Third, the transductive learning process also appeared to impact the sample predictions suffering from border uncertainty, where the predicted distribution fell in close proximity to the classification threshold. Compared to the standard GP baseline model, the transductive learning model shifted the domain, such that most of these sample predictions could be interpolated, which further improved model performance. Finally, we noted that the incorporation of uncertainty in the training of the transductive learning model resulted in a substantial reduction in selected features and model complexity, compared with the standard GP approach to model training. While lower model complexity would suggest greater generalizability, further studies are likely needed to confirm this assertion.

Using a GP model offers the flexibility of identifying nonlinear relationships between input and output variables. Furthermore, as described above, the GP model is capable of generating a probability distribution, rather than point estimates, which allows for direct quantification of both predictive uncertainty and predictive accuracy. These technical capabilities provide improved genetic prediction data, or other biological prediction data and corresponding predictive uncertainty data, which can be used to better guide follow-on clinical decision making.

The standard GP model measures, but does not explicitly incorporate, predictive uncertainties for model optimization during the training phase of development. As such, the standard GP model can measure predictive uncertainty, but is built without considering the predictive uncertainty.

As an illustrative example of a GP model, let {x₁, . . . x_{N}} be the input variables (i.e., texture features) corresponding to N samples. GP assumes a set of random functions corresponding to these samples, {ƒ(x₁), . . . , ƒ(x_{N})}. This is called a Gaussian process because any subset of {ƒ(x₁), . . . , ƒ(x_{N})} follows a joint Gaussian distribution with zero mean and the covariance between two samples xᵢ and xⱼ computed by a kernel function, K(xᵢ,xⱼ). The input variables are linked with the output (e.g., transformed copy number variant (“CNV”) data) by yᵢ=ƒ(xᵢ)+εᵢ, where εᵢ˜N(0,σ²) is a Gaussian noise.

Given a training dataset {X, Y} where X is a matrix containing the input variables of all training samples and Y is a vector containing the output variables of these samples, the predictive distribution for a new test sample x* can be given as,

ƒ(x*)˜N(μ*,σ*₂)

where

μ*=K(x*,X)(K(X,X)+σ²I)⁻¹Y

σ*²=K(x*,x*)−K(x*,X)(K(X,X)±σ²I)⁻¹K(x*,X)^{T}

While the predictive mean can be used as a point estimate of the prediction, the variance provides additional information about the uncertainty of the prediction. Furthermore, using this predictive distribution, one can test the hypothesis that a prediction is greater or less than a cutoff of interest. For instance, as a non-limiting example, it may be desirable to know if the prediction of the CNV of a sample is greater than 3.5 (or another number that may be considered as amplification). A small p-value (e.g., less than 0.5) of the hypothesis testing can imply prediction with certainty.

As described above, the radiogenomics model is designed to not only maximize predictive accuracy, but also minimize the predictive uncertainty. To this end, a transductive learning component can be added to the standard GP model to use the predictive uncertainty quantified by the GP model during an iterative model training process to minimize uncertainty on subsequent predictions. As an example, this model employs transductive learning to perform feature selection in an iterative, stepwise fashion to prioritize the minimization of average predictive uncertainty during model training.

Although a typical supervised learning model is trained using labeled samples (i.e., samples with both input and output variables available), some machine learning algorithms have been developed to additionally incorporate unlabeled samples (i.e., samples with only input variables available). These algorithms belong to a machine learning subfield called transductive learning or semi-supervised learning. Transductive learning can be beneficial when sample labeling is costly or labor-intensive, which results in a limited sample size.

While the standard GP model described above utilizes labeled samples, a transductive GP model can use both labeled and unlabeled samples. As an illustrative example, let {X_{L}, Y_{L}} and {X_{U}} be the sets of labeled and unlabeled samples used in training, respectively. A transductive GP model first generates predictions for the unlabeled samples by applying the standard GP to the labeled samples, Ŷ_{U}. Here, Ŷ_{U} contains the means of the predictive distributions. Then, a combined dataset {X_{L}, Y_{L}}∪{X_{U}, Ŷ_{U}} can be used as the training dataset to generate a predictive distribution for each new test sample x*, i.e.,

$${f}_{t r a n} ( {x}^{*} ) ∼ N ( {μ}_{tran}^{*} , {{σ}_{tran}^{*}}^{2} )$$

$$with$$

$${μ}_{tran}^{*} = {{K}_{t r a n} ( K ( {X}_{L} , {X}_{L} ) + {σ}^{2} I )}^{- 1} {Y}_{L}$$

$${σ}^{{*}_{2}} = K ( {x}^{*} , {x}^{*} ) - {( \begin{matrix}K ( {x}^{*} , {X}_{L} ) \\ K ( {x}^{*} , {X}_{U} )\end{matrix} )}^{T} {( \begin{matrix}( K ( {X}_{L} , {X}_{L} ) + {σ}^{2} I ) & K ( {X}_{L} , {X}_{U} ) \\ {K ( {X}_{L} , {X}_{U} )}^{T} & ( K ( {X}_{U} , {X}_{U} ) + {σ}^{2} I )\end{matrix} )}^{- 1} ( \begin{matrix}K ( {x}^{*} , {X}_{L} ) \\ K ( {x}^{*} , {X}_{U} )\end{matrix} )$$

$${K}_{t r a n} = K ( {x}^{*} , {X}_{L} ) + K ( {x}^{*} , {X}_{U} ) {( K ( {X}_{U} , {X}_{U} ) + {σ}^{2} I )}^{- 1} K ( {X}_{U} , {X}_{L} ) .$$

To decide which unlabeled samples to include in transductive GP, a self-similarity (i.e., the similarity between the unlabeled and test samples) can be used. As one example based on this consideration, the eight neighbors of the test sample on the image can be included as the unlabeled samples. The labeled samples are those from other patients (i.e., other than the patient where the test sample is from), which are the same as the samples used in GP. Because transductive GP also generates a predictive distribution, the same approach as that described above for GP can be used to quantify the uncertainty of the prediction.

The systems and methods described above can be generalized using a knowledge-infused global-local (“KGL”) data fusion model.

As described above, transductive learning (e.g., semi-supervised learning (“SSL”) can be used in situations where labeled samples are scarce, but unlabeled samples are available in a large quantity. Supervised learning models often utilize only labeled samples to build a predictive model, whereas SSL can additionally leverage the unlabeled samples. Within the context of radiogenomics, local data with direct measurement for the variable of interest (e.g., biopsy samples and survey samples) are labeled and scarce, whereas image data (e.g., medical images such as magnetic resonance images) with indirect measurements are usually unlabeled, but available global-wide.

SSL algorithms can fall into several main categories. As one example, SSL can implement self-training, which is a type of wrapper algorithm that repeatedly adds those unlabeled samples predicted with the highest confidence to the training set. As another example, SSL can implement co-training, which is an extension of self-training that leverages two views of the data. Co-training generally assumes that there are two separate datasets that contain conditionally independent feature sets. Two classifiers are built on the two datasets but with information exchange with each other. As still another example, SSL can implement low-density separation, which aims to find the decision boundary in low density regions in the feature space based on labeled and unlabeled samples. As yet another example, SSL can implement graph-based models that define a graph in which nodes represent labeled and unlabeled samples and edges reflect the similarity between nodes. Label smoothness can be assumed over the graph to allow label diffusion to unlabeled samples.

As also described above, a GP model is a type of Bayesian non-parametric kernel-based probabilistic model. Compared to other predictive models, GP has some unique aspects. As one example, GP makes few assumptions about the shape of the estimator function beyond the assumptions associated with the choice of the covariance function. As another example, the GP model is inherently probabilistic in nature. GP can generate a predictive distribution for the response variable based on features, instead of just a point estimator of the prediction. This allows for uncertainty quantification and more informed decision making based on the prediction result, as described above.

The systems and methods described in the present disclosure can implement models other than the GP model. The GP model is advantageous because it is non-parametric, non-linear, and has the capacity for generating a predictive probability distribution for a variable of interest. This allows for uncertainty quantification and reduction, as described above.

In some embodiments, genetic prediction data can be generated using a KGL data fusion predictive model that can simultaneously leverage labeled, local direct measurement data and unlabeled, global image data; can reduce the uncertainty of the prediction; and can integrate domain knowledge with data-driven model training.

An example KGL data fusion model is now described. Let ƒ be a random variable corresponding to an input vector x. As noted above, a GP is a collection of these random variables, any finite number of which have a joint Gaussian distribution. Consider a set that includes L labeled samples, {xᵢ, yᵢ}ᵢ₌₁^{L}, and an unlabeled sample, x*∈{xᵢ}_{i=L+1}^{L+U}. The joint Gaussian distribution of this set is:

$$[ \begin{matrix}f \\ {f}^{*}\end{matrix} ] = N ( 0 , [ \begin{matrix}K ( {X}_{L} , {X}_{L} ) & K ( {X}_{L} , {x}^{*} ) \\ {K ( {X}_{L} , {x}^{*} )}^{T} & K ( {x}^{*} , {x}^{*} )\end{matrix} ] ) , \qquad ( 1 ) ;$$

where K contains covariances between the corresponding samples, computed based on the input variables using kernels. Furthermore, introducing the noise term, the joint distribution of response variables corresponding to the labeled and unlabeled samples is:

$$[ \begin{matrix}{y}_{L} \\ {y}^{*}\end{matrix} ] = [ \begin{matrix}f \\ {f}^{*}\end{matrix} ] + {σ}^{2} {I}_{( L + 1 ) \times ( L + 1 ) ,} \qquad ( 2 ) .$$

To predict the response of the unlabeled sample x*, the predictive distribution of ƒ* can be obtained by combining Eqns. (1) and (2), i.e.,

ƒ*|X_{L},y_{L},x*˜N(μ*,σ*²) (3);

where

μ*=K(X_{L},x*)^{T}(K(X_{L},X_{L})+σ²I_{L×L})⁻¹y_{L}

σ*²=K(x*,x*)−K(X_{L},x*)^{T}(K(X_{L},X_{L})+σ²I_{L×L})⁻¹K(X_{L},x*).

Eqn. (3) contains parameters to be estimated, including parameters in the kernel function and σ². Let θ be the set of all parameters. Then, θ can be estimated by maximizing the marginal likelihood of the labeled samples, i.e.,

min_{θ}l(θ)=min_{θ}−log p(y_{L}|X_{L},θ) (4).

For the KGL data fusion model, again let {xᵢ,yᵢ}ᵢ₌₁^{L} be L labeled samples, and let {xᵢ}_{i=L+1}^{L+U} be U unlabeled samples (e.g., image features extracted from U locations of an area of interest, such as a tumor, a forest, a developing world). Then, let y∈ [symbol] be the measurement of a variable of interest (e.g., a molecular marker, fire risk, poverty level). The objective is to build a model using {xᵢ,yᵢ}ᵢ₌₁^{L} and {xᵢ}_{i=L+1}^{L+U} together with domain knowledge in order to predict {ŷᵢ}_{i=L+1}^{L+U}.

The advantage of a GP model is that it can produce a predictive distribution, in which the predictive variance σ*² reflects the certainty/uncertainty of the prediction. Also note that σ*² can be computed using only the image features of an unlabeled sample. This leads to an SSL extension of the GP:

$$\begin{matrix}{min}_{θ} \frac{1}{L} l ( θ ) ; & ( 5 ) \\ such that \frac{1}{U} {\sum }_{i = L + 1}^{L + U} Var ( {f}_{i} ) \leq t , & ( 6 ) ;\end{matrix}$$

which minimizes the average negative marginal likelihood under a constraint that upper-bounds the sum of predictive variances on unlabeled samples. Compared with the supervised learning model in (4), the SSL-based model considers uncertainty reduction in predicting the unlabeled samples, not just maximizing the likelihood of labeled samples.

Furthermore, considering that domain knowledge may exist, additional constraints to can be added on the predictive means of unlabeled samples in Eqn. (6), i.e.,

$$\begin{matrix}{min}_{θ} \frac{1}{L} l ( θ ) ; & ( 7 ) \\ such that \frac{1}{U} {\sum }_{i = L + 1}^{L + U} Var ( {f}_{i} ) \leq t , & ( 8 ) ; \\ g ( E ( {f}_{L + 1} ) , . . . , E ( {f}_{L + U} ) ) \leq ξ , & ( 9 ) ; \\ ξ \geq 0 , & ( 10 ) ; \\ {ξ}^{T} 1 \leq ϵ , & ( 11 ) .\end{matrix}$$

where 1 is a vector of m ones. g(⋅) contains m different functions, g₁(⋅), . . . , gₘ(⋅). Each gⱼ(⋅) is a function of the predictive means of unlabeled samples, j=1, . . . , m, and ξ=(ξ₁, . . . , ξₘ) contains the upper bounds of these functions. A special case is when m=1. In that instance, Eqn. (9) reduces a single function of g(E(ƒ_{L+1}), . . . , E(ƒ_{L+U}))≤ξ. Sometimes, a single function may not be enough to represent different kinds of domain knowledge. Therefore, a general notation in Eqn. (9) can be used to allow for m functions of different forms. Also, it is noted that when the domain knowledge is in the form of an equation but not an inequality, i.e., g(E(ƒ_{L+1}), . . . , E(ƒ_{L+U}))=ξ, the equation can be represented by two inequalities of g₁(E(ƒ_{L+1}), . . . , E(ƒ_{L+U}))≤ξ and −g₂(E(ƒ_{L+1}), . . . , E(ƒ_{L+U}))≤ξ which can be added to the constraint set in Eqn. (9).

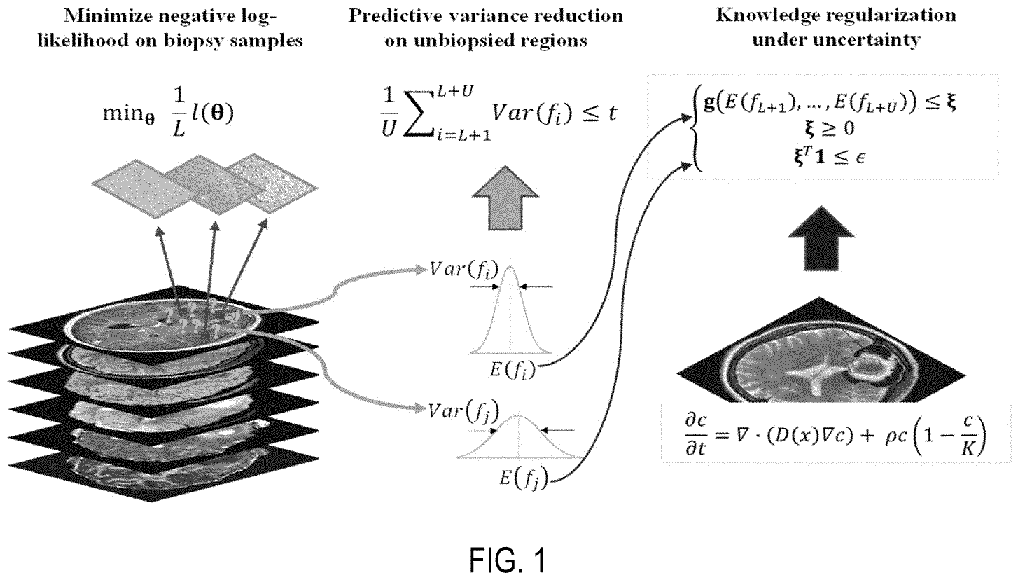

Additionally, it can be considered that domain knowledge may not always be completely accurate. To accommodate this uncertainty, slack variables can be used to specify the constraints corresponding to domain knowledge, as shown in Eqns. (9)-(11). The parameter, E, controls the extent to which the domain knowledge constraints can be violated. This adds the flexibility of allowing some small violations of these constraints. FIG. 1 shows a graphical illustration of an example constrained optimization framework for KGL.

As a non-limiting example, to solve the optimization problem in Eqns. (7)-(11), the corresponding Lagrangian function can be written as,

$$ℒ = \frac{1}{L} l ( θ ) + {α}_{1} ( \frac{1}{U} {Σ}_{i = L + 1}^{L + U} Var ( {f}_{i} ) - t ) + {Σ}_{j = 1}^{m} {μ}_{j} ( {g}_{j} ( \cdot ) - {ξ}_{j} ) - {Σ}_{j = 1}^{m} {v}_{j} {ξ}_{j} + {α}_{2} ( {Σ}_{j = 1}^{m} {ξ}_{j} - ∈ ) , \qquad ( 12 ) ;$$

with Lagrange multipliers μ=(μ₁, . . . , μₘ), v=(v₁, . . . , vₘ), α₁∈ [symbol] and a₂∈ [symbol] , and gⱼ(⋅) used to represent gⱼ(E(ƒ_{L+1}), . . . , E(ƒ_{L+U})) for notation simplicity. Then, in this example, the optimal solution of the primal problem in Eqns. (7)-(11) is equivalent to the solution of the following optimization:

$$\begin{matrix}{inf}_{θ , ξ} {sup}_{μ \geq 0 , v \geq 0 , {α}_{1} \geq 0 , {α}_{2} \geq 0} ℒ . & ( 13 ) . \\ Let , & \\ {ℒ}^{′} = \frac{1}{L} l ( θ ) + {λ}_{1} ( \frac{1}{U} {Σ}_{i = L + 1}^{L + U} Var ( {f}_{i} ) ) + {Σ}_{j = 1}^{m} {μ}_{j} ( {g}_{j} ( \cdot ) - {ξ}_{j} ) - {Σ}_{j = 1}^{m} {v}_{j} {ξ}_{j} + {λ}_{2} ( {Σ}_{j = 1}^{m} {ξ}_{j} ) , & ( 14 ) ;\end{matrix}$$

where λ₁ and λ₂ are tuning parameters. Then, the optimal solution of Eqn. (13) is equal to that of inf_{θ,ξ}sup_{μ≥0,v≥0} [symbol] ′. Accordingly, Eqn. (13) can be further simplified as:

inf_{θ,ξ}sup_{μ≥0,v≥0} [symbol] ′. (15).

Since [symbol] ′ is a convex function of ξⱼ, μ, v (non-convex of θ), Equation (15) is equivalent to the following:

inf_{θ}sup_{μ≥0,v≥0}inf_{ξ} [symbol] ′. (16).

Focusing on the inner minimization in Eqn. (16), the minimizer of ξⱼ should satisfy the following:

$$\frac{\partial {ℒ}^{′}}{\partial {ξ}_{j}} = {λ}_{2} - {μ}_{j} - {v}_{j} = 0 , j = 1 , . . . , m ; \qquad ( 17 )$$

From Eqn. (17), vⱼ can be written as vⱼ=λ₂−μⱼ. Inserting this into Eqn. (16) yields the following:

$$\begin{matrix} {inf}_{θ} {sup}_{μ \geq 0} 𝒥 ( {μ}_{j} ; j = 1 , . . . m ) ; & ( 18 ) \\ such that 0 \leq {μ}_{j} \leq {λ}_{2} , j = 1 , . . . , m ; & ( 19 ) \\ where & \\ 𝒥 ( {u}_{j} ; j = 1 , . . . , m ) = \frac{1}{L} l ( θ ) + {λ}_{1} ( {\sum }_{i = L + 1}^{L + U} Var ( {f}_{i} ) ) + {\sum }_{j = 1}^{m} {μ}_{j} {g}_{j} ( \cdot ) . & ( 20 )\end{matrix}$$

The solution of the inner maximization of (18) with (19) is,

$${μ}_{j} = \{ \begin{matrix}{λ}_{2} , & if {g}_{j} ( \cdot ) > 0 \\ any value in [ 0 , {λ}_{2} ] , & if {g}_{j} ( \cdot ) = 0 \\ 0 , & if {g}_{j} ( \cdot ) < 0\end{matrix} . \qquad ( 21 )$$

Then, in this example, the final objective function becomes:

$${inf}_{θ} L ( θ ) = {inf}_{θ} \frac{1}{L} l ( θ ) + {λ}_{1} ( {\sum }_{i = L + 1}^{L + U} Var ( {f}_{i} ) ) + {λ}_{2} {\sum }_{j = 1}^{m} {g}_{j} ( \cdot ) I ( {g}_{j} ( \cdot ) > 0 ) , \qquad ( 22 ) .$$

The gradient of the objective function in Eqn. (22) can be written as,

$$∇ {L}_{θ} = \frac{1}{L} ∇ l ( θ ) + {λ}_{1} ( {\sum }_{i = L + 1}^{L + U} ∇ Var ( {f}_{i} ) ) + {λ}_{2} {\sum }_{j = 1}^{m} ∇ {g}_{j} ( \cdot ) I ( {g}_{j} ( \cdot ) > 0 ) . \qquad ( 23 ) .$$

As a non-limiting example, the above-described optimization can be solved by a gradient descent algorithm, or other suitable algorithm.

Note that the optimization in Eqn. (22) simultaneously balances three aspects: maximizing the average marginal likelihood on labeled samples (recall that l(θ) is the negative marginal likelihood as defined in (4)); minimizing the predictive variances/uncertainty on unlabeled samples; and optimizing the consistency with domain knowledge. The last term in (22), I(gⱼ(⋅)>0), is an indicator function that takes the value of one if gⱼ(⋅)>0 and zero otherwise. In the KGL formulation in Eqns. (7)-(11), the consistency with domain knowledge is imposed by having the constraints of gⱼ(⋅)<ξⱼ, ξⱼ≥0, j=1, . . . , m, where m different types of domain knowledge are considered. The utility of the indicator function is to find which subset of these constraints should be satisfied. This is the subset corresponding to gⱼ(⋅)≤0 or equivalently I(gⱼ(⋅)>0)=0. For the remaining constraints corresponding to gⱼ(⋅)>0 or equivalently I(gⱼ(⋅)>0)=1, the model will try to satisfy these constraints as much as possible, but is traded off with the first two terms in the optimization (i.e., some degree of violations for these constraints is allowed). The advantageous aspect of the KGL model is that it does not require pre-specifying which subset of constraints must be satisfied and which must not, nor does it require specifying how much violation is allowed. All these aspects of the KGL model can be automatically resolved through solving the optimization problem.

To incorporate domain knowledge in probabilistic models, a common approach is to specify a prior of the model M that reflects the domain knowledge, i.e., π(M). This prior is then integrated with the data likelihood p(D|M) using the Bayes' rule to obtain the posterior p(M|D). In this approach, domain knowledge does not directly impact or regularize the final model estimate, but indirectly affects the model through prior specification. Due to the indirect nature of this influence, the final model estimate may not fully comply with the knowledge.

In some applications, it may be preferred that domain knowledge can be used to directly regularize the posterior. In these instances, a posterior regularization (“PostReg”) framework can be utilized. In general, PostReg uses a variational distribution q(M|D) to approximate the posterior p(M|D), while at the same time regularizing q(M|D) according to domain knowledge. That is, PostReg aims to find the solution q*(M|D) for the following optimization:

inf [symbol] KL(q(M|D)∥p(M|D))+Ω(q(M|D)) (24).

In this example, the first term is the Kullback-Leibler (KL)-divergence, defined as the expected log-difference between the posterior and approximate distributions. The term, Ω(⋅), is a function of the approximate distribution, which regularizes this distribution to comply with domain knowledge. Because of the regularization effect, q(M|D) is made close to the posterior, p(M|D), while at the same time being consistent with the domain knowledge. [symbol] _{prob} denotes a proper variational family of distributions. The PostReg optimization in (24) is a general formulation that can be realized for different models, such as latent variable models, multi-view learning, and infinite support vector machines.

Solving the example optimization in Eqns. (7)-(11) can be similar to solving a specific form of the PostReg optimization. In this specific form, the choice of the regularizer Ω(q(M|D)) corresponds to variance minimization and consistency with domain knowledge in expectation.

For instance, the optimization in Eqns. (7)-(11) can be similar to a PostReg optimization taking the following form:

inf [symbol] KL(q(M|D)∥P(M|D))+Ω(q(M|D)), (25);

with the following notations: M=(ƒ, θ) is the model; D=({xᵢ,yᵢ}ᵢ₌₁^{L}, {xᵢ}_{i=L+1}^{L+U}) is the data; [symbol] _{prob}={q|q(ƒ,θD)=p(ƒ|θ,D)δ_{θ̅}(θ|D), θ̅∈Θ} is a variational family of distributions where q(ƒ|θ,D)=p(ƒ|θ,D) and q(θ| [symbol] )=δ_{θ̅}(θ|D) which can be a Dirac delta function centered on θ in the parameter space θ; Ω(q(ƒ,θ|D)), denoted by a simple form of Ω(q) hereafter, can be given by

$$Ω ( q ) = {inf}_{t , ξ} \{ ( {λ}_{1} t + {λ}_{2} {\sum }_{j = 1}^{m} {ξ}_{j} ) \vert \begin{matrix}\begin{matrix}\frac{1}{U} {\sum }_{i = L + 1}^{L + U} ( {\int }_{f , θ} q \times ( f ( {x}_{i} ) - \\ { {E}_{q} [ f ( {x}_{i} ) ] )}^{2} d η ( f , θ ) ) \leq t ;\end{matrix} \\ \begin{matrix}g ( {\int }_{f , θ} q \times f ( {x}_{L + 1} ) d η ( f , θ ) , . . . , \\ {\int }_{f , θ} q \times f ( {x}_{L + U} ) d η ( f , θ ) ) \leq ξ ;\end{matrix} \\ ξ \geq 0\end{matrix} \} . \qquad ( 26 )$$

By demonstrating that KGL is a specific instance within the general PostReg framework, the following insights can be gained. As one example, it can be seen that domain knowledge is imposed to regularize the posterior of the model (not the prior nor by any other means). As another example, KGL provides a realization of the general PostReg framework and enriches the problem set PostReg can potentially address. Although PostReg provides a theoretical framework, the KGL models described in the present disclosure provide a practical utility of using the concept of PostReg to integrate local data, global data, and domain knowledge for spatial estimation.

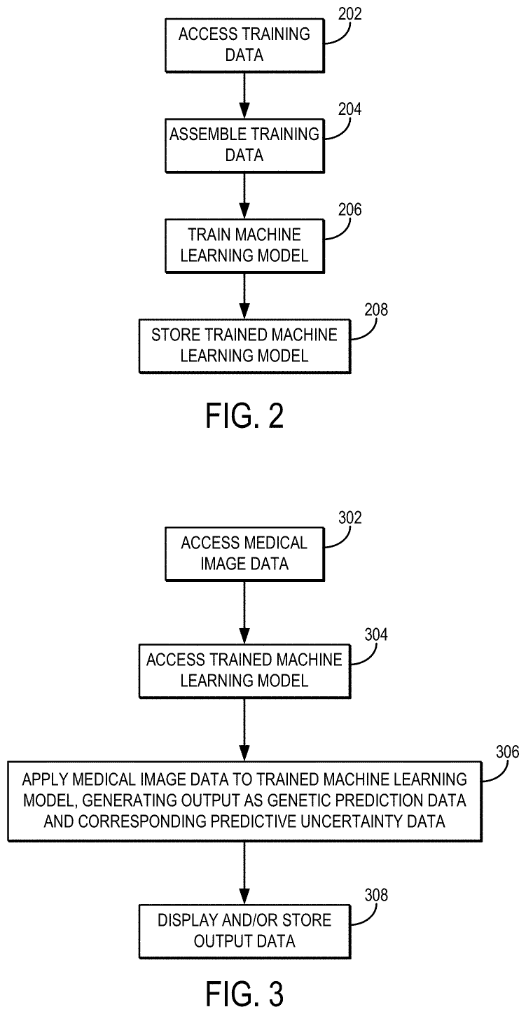

FIGS. 2 and 3 show flowcharts that respectively illustrate example methods for training a predictive model and implementing the trained model to generate genetic prediction data, or other biological prediction data, which includes predictive uncertainty data that indicate an uncertainty measure of each prediction. For instance, biological prediction data other than genetic prediction data can be output, including other tissue characteristics, such as molecular pathways, quantity and location of tumor cell density, and non-tumoral cells such as immune/stromal cell types. Advantageously, these additional biological predictions can be generated based on the same training data used to train the model (e.g., image-localized biopsies). These output data can then be used by a clinician in the clinical decision making process to determine the next best step in treatment.

Referring first to FIG. 2, a flowchart is illustrated as setting forth the steps of an example method for training one or more machine learning models and/or algorithms on training data. Particularly, the one or more machine learning models and/or algorithms are trained to receive input as medical image data and generate output as genetic prediction data, or other biological prediction data, which include predictive uncertainty data that indicate an uncertainty in each genetic and/or biological prediction.

In general, the machine learning model and/or algorithm can be a GP model based on or otherwise implementing a Gaussian process. As one non-limiting example, the machine learning model may be a transductive learning GP model, as described above. Alternatively, the machine learning model can be a KGL model, such as the KGL model described above. The models have the advantage of flexibility in identifying nonlinear relationships between input and output variables. Further still, the models can generate a probability distribution for each prediction, which quantifies the uncertainty of the prediction. This capability is advantageous for using the prediction result to inform the clinical decision making process. Because the models generate probability distributions (rather than point estimates), this allows for direct quantification of both predictive uncertainty and predictive accuracy by using the predictive variance or standard deviation of the distribution and the predictive mean in comparison with the true genetic or other biological feature status, respectively.

The method includes accessing training data with a computer system, as indicated at step 202. Accessing the training data may include retrieving such data from a memory or other suitable data storage device or medium. Alternatively, accessing the training data may include acquiring such data and transferring or otherwise communicating the data to the computer system.

In general, the training data can include spatially matched medical image data (e.g., magnetic resonance images) and image-localized genetic or other biological data. These data can be used to build a predictive model for the probability distributions of genetic feature and/or biological marker amplification for each biopsy specimen. In this way, the training data may include local data as labeled genetic or other biological data and global data as medical image data.

As a non-limiting example, the training data can include medical image data as magnetic resonance images. The magnetic resonance images can be any suitable magnetic resonance image, including but not limited to T1-weighted images (T1W), T1-weighted images with contrast-enhancement (T1W+C), T2-weighted images (T2 W), diffusion weighted images, perfusion weighted images, and so on. Preferably, the medical image data are multiparametric images representing a plurality of different image contrasts and/or modalities. For instance, the medical image data may be multiparametric magnetic resonance images representative of two or more different images contrasts (e.g., T1W, T1W+C, T2W, diffusion weighting, perfusion weighting).

The training data can also include medical image data as parametric maps computed or otherwise derived from medical images, such as magnetic resonance images. For instance, the training data may also include diffusion parametric maps, such as apparent diffusion coefficient (“ADC”) maps and/or diffusion tensor imaging (“DTI”) parameter maps, such as maps of mean diffusivity (“MD”) and/or fractional anisotropy (“FA”). Additionally or alternatively, the parametric maps may include perfusion parameter maps, such as relative cerebral blood volume (“rCBV”) maps, relative cerebral blood flow (“rCBF”) maps, mean transit time (“MTT”) maps, and maps of other such perfusion parameters.

The training data can also include image-localized genetic and/or other biological marker data, which may include genetic and/or other biological data derived or otherwise determined from image-localized biopsies, such as stereotactic biopsies. For instance, the image-localized biopsies may be tissue specimens from tumors or other anatomical ROIs that have been obtained using stereotactic surgical localization. Within the context of tissue specimens obtained from a tumor, the biopsy targets can be separated from both enhancing core and non-enhancing abnormalities (e.g., non-enhancing T2W abnormalities) in pseudorandom fashion. As a non-limiting example, the biopsy targets can be separated from the enhancing core and non-enhancing abnormalities by at least 1 cm. The biopsy locations can be recorded, such as via screen capture, to allow subsequent co-registration with multiparametric medical image datasets.

In one non-limiting example implementation, tissue specimens were obtained and obtained and processed as follows. Tissue specimens (target volume of 125 mg) were flash frozen in liquid nitrogen within 1-2 min from collection in the operating suite and stored in −80° C. freezer until subsequent processing. Tissue was retrieved from the freezer and embedded frozen in optimal cutting temperature (“OCT”) compound. Tissue was cut at 4 μm sections in a −20° C. cryostat (Microm-HM-550) utilizing microtome blade. Tissue sections were stained with hematoxylin and eosin (H&E) for neuropathology review to ensure adequate tumor content (50%).

DNA isolation was performed and copy number variant (“CNV”) was determined for all tissue samples using array comparative genomic hybridization (“aCGH”) and exome sequencing. This included application of CNV detection to whole genome long insert sequencing data and exome sequencing. Briefly, tumor-normal whole exome sequencing was performed. DNA from fresh frozen tumor tissue and whole blood (for constitutional DNA analysis) samples were extracted and QC was performed using DNAeasy Blood and Tissue Kit (Qiagen #69504). Tumor-normal whole exome sequencing was performed with the Agilent Library Prep Kit RNA libraries. Sequencing was performed using the Illumina HiSeq 4000 with 100 bp paired-end reads. Fastq files were aligned with BWA 0.6.2 to GRCh37.62 and the SAM output were converted to a sorted BAM file using SAMtools 0.1.18. Lane level samples BAMs were then merged with Picard 1.65 if they were sequenced across multiple lanes. Comparative variant calling for exome data was conducted with Seurat. Copy number detection was applied to the whole exome sequence. Briefly, copy number detection was based on a log 2 comparison of normalized physical coverage (or clonal coverage) across tumor and normal whole exome sequencing data. Normal and tumor physical coverage was then normalized, smoothed and filtered for highly repetitive regions prior to calculating the log 2 comparison. To quantify the copy number aberrations, CNV score was calculated based on the intensity of copy number change (log ratio).

Additionally or alternatively, the method can include assembling training data from medical image data and image-localized genetic and/or other biological marker data using a computer system, as indicated at step 204. This step may include assembling the medical image data and image-localized genetic and/or other biological marker data into an appropriate data structure on which the machine learning model and/or algorithm can be trained. Assembling the training data may include assembling feature data, segmented medical image data, and other relevant data. For instance, assembling the training data may include generating labeled data and including the labeled data in the training data. Labeled data may include labeled medical images, labeled genetic and/or other biological marker data, segmented medical images, or other relevant data that have been labeled as belonging to, or otherwise being associated with, one or more different classifications or categories. For instance, labeled data may include medical images and/or segmented medical images that have been labeled based on the image-localized genetic and/or other biological marker data. The labeled data may include data that are classified on a voxel-by-voxel basis, or a regional or larger volume basis.

In a non-limiting example, regions-of-interest (“ROIs”) can be generated using a defined neighborhood size (e.g., a neighborhood measuring 8×8×1 voxels) for each corresponding biopsy location. From each ROI, texture analysis can be performed to extract various different texture features from each sliding window. For example, the texture features may include measurements of first-order statistics from raw image signals (18 features): mean (M) and standard deviation (SD) of gray-level intensities, Energy, Total Energy, Entropy, Minimum, 10th percentile, 90th percentile, Maximum, Median, Interquartile Range, Range, Mean Absolute Deviation (MAD), Robust Mean Absolute Deviation (rMAD), Root Mean Squared (RMS), Skewness, Kurtosis, Uniformity. Intensity values within each window can be mapped onto the range of 0-255 to help standardize intensities and reduce effects of intensity non-uniformity on features extracted during subsequent texture analysis. Texture analysis can be performed using two separate but complementary texture algorithms: gray level co-occurrence matrix (“GLCM”) and Gabor Filters (“GF”). The output from the texture analysis pipeline can be stored as a feature vector from each sliding window, where as one non-limiting example the feature vector can be composed of 56 features across each of the 6 MRI contrasts, for a total of 336 (6*56) features.

Appropriate feature selection can be implemented to reduce the risk of overfitting when the input variables are high-dimensional. As a non-limiting example, a forward stepwise selection can be used, which starts with an empty feature set and adds one feature at each step that maximally improves a pre-defined criterion until no more improvement can be achieved. To avoid overfitting, the accuracy computed on a validation set can be used as an evaluation criterion; when the sample size is limited, cross-validation accuracy can be adopted. For example, a leave-one-patient-out cross validation accuracy can be adopted to be consistent with the natural grouping of samples. The accuracy can be computed by comparing the true and predicted CNVs; a match is counted when both are on the same side of the amplification threshold, such as 3.5 (i.e., both being amplified or non-amplified). The same forward stepwise selection procedure can be used for GP, transductive GP, and/or KGL models. Because transductive GP and MGL models significantly reduce the prediction variance, feature selection can benefit from minimizing leave-one-patient-out cross validation uncertainty as the criterion instead of maximizing the accuracy. Specifically, the p-value can be computed for each prediction that reflects the uncertainty (the smaller the p-value, the more certain of the prediction) and features can be selected to minimize the leave-one-patient-out cross validation p-value.

Referring still to FIG. 2, one or more machine learning models and/or algorithms are then trained on the training data, as indicated at step 206. In general, the machine learning model can be trained by optimizing model parameters based on minimizing a loss function. As one non-limiting example, the loss function may be a mean squared error loss function.

Training a machine learning model may include initializing the model, such as by computing, estimating, or otherwise selecting initial model parameters. Training data can then be input to the initialized machine learning model, generating output as genetic and/or other biological marker data and predictive uncertainty data that indicate an uncertainty in those genetic and/or other biological marker predictions. The quality of the output data can then be evaluated, such as by passing the output data to the loss function to compute an error. The current machine learning model can then be updated based on the calculated error (e.g., using backpropagation methods based on the calculated error).

As described above, a transductive learning or semi-supervised learning approach can be implemented. In these instances, the training data may include both labeled data and unlabeled data. A transductive learning-based model first generates predictions for the unlabeled samples by applying model to the labeled samples, which may contain the means of predictive distributions. Then, a combined dataset of labeled and unlabeled samples can be used as the training dataset to generate a predictive distribution for each new test sample.

The current machine learning model can be updated by updating the model parameters in order to minimize the loss according to the loss function. When the error has been minimized (e.g., by determining whether an error threshold or other stopping criterion has been satisfied), the current model and its associated model parameters represent the trained machine learning model.

The one or more trained neural networks are then stored for later use, as indicated at step 208. Storing the neural network(s) may include storing network parameters (e.g., weights, biases, or both), which have been computed or otherwise estimated by training the neural network(s) on the training data. Storing the trained neural network(s) may also include storing the particular neural network architecture to be implemented. For instance, data pertaining to the layers in the neural network architecture (e.g., number of layers, type of layers, ordering of layers, connections between layers, hyperparameters for layers) may be stored.

Referring now to FIG. 3, a flowchart is illustrated as setting forth the steps of an example method for generating genetic prediction data, or other biological prediction data and corresponding predictive uncertainty data using a suitably trained machine learning model and/or algorithm.

The method includes accessing medical image data with a computer system, as indicated at step 302. Accessing the medical image data may include retrieving such data from a memory or other suitable data storage device or medium. Alternatively, accessing the medical image data may include acquiring such data with an imaging system, such as an MRI system, and transferring or otherwise communicating the data to the computer system, which may be a part of the imaging system. In still other instances, accessing the medical image data may include retrieving and/or acquiring medical images and then computing parameter maps, features, or other data from those medical images and providing those with the medical images as input data.

A trained machine learning model and/or algorithm is then accessed with the computer system, as indicated at step 304. Accessing the trained machine learning model may include accessing model parameters that have been optimized or otherwise estimated by training the machine learning model on training data. In some instances, retrieving the machine learning model can also include retrieving, constructing, or otherwise accessing the particular machine learning model to be implemented.

In general, the machine learning model is trained, or has been trained, on training data in order to predict genetic and/or other biological marker data from medical imaging data and also to quantify the uncertainty of those predictions.

The medical image data are then input to the one or more trained machine learning models, generating output as genetic prediction data, or other biological prediction data and corresponding predictive uncertainty data, as indicated at step 306. For example, the genetic prediction data, or other biological prediction data may include feature maps associated with genetic markers and/or other biological markers or quantities, such as other tissue characteristics, including molecular pathways, quantity and location of tumor cell density, and non-tumoral cells such as immune/stromal cell types. The feature maps may depict the spatial distribution or spatial patterns of features, statistics, or other parameters associated with one or more genetic markers and/or other biological markers. The output data also include predictive uncertainty data, which indicate the quantified uncertainty in the predictions contained in the genetic prediction data, or other biological prediction data.

As one example, the feature maps may indicate the local probability for a particular classification (i.e., the probability that a voxel belongs to a particular class), such as a particular genetic marker and/or other biological marker. For instance, the feature maps may be radiogenomics color maps that depict spatial patterns of genetic predictions and/or other biological markers in an anatomical ROI. As one non-limiting example, the radiogenomics color maps can depict the spatial pattern of genetic marker amplification (e.g., EGFR amplification) predicted in an anatomical ROI. In these instances, the radiogenomics color maps can resolve regional heterogeneity of genetic marker (e.g., EGFR) amplification status.

As another example, the feature maps may indicate a quantification of the predictive uncertainty in the genetic prediction data, or other biological prediction data (e.g., by assigning a particular predictive uncertainty value to each voxel in the feature map).

The output genetic prediction and/or other biological marker data and predictive uncertainty data generated by inputting the medical image data to the trained machine learning model and/or algorithm can then be displayed to a user, stored for later use or further processing, or both, as indicated at step 308.

In an example study, the described methods were implemented to develop a model in the setting of glioblastoma multiforme (“GBM”), which presents particular challenges for individualized oncology due to its profound intratumoral heterogeneity and likelihood for tissue sampling errors. The confounds of intratumoral heterogeneity are addressed by utilizing an annotated dataset of image-localized biopsies with spatially matched MRI measurements from a cohort of untreated GBM patients. Predictions of EGFR amplification status are generated as an example output, as this serves as a common therapeutic target for many clinically available drugs. A progression of optimization steps that not only quantify, but also minimize predictive uncertainty, are implemented. Advantageously, the output data provide a pathway to clinically integrating reliable radiogenomics predictions as part of decision support within the paradigm of individualized oncology.

A CNV threshold of 3.5 was used to classify each biopsy sample as EGFR normal (CNV<3.5) versus EGFR amplified (CNV>3.5) as a statistically conservative approach to differentiating amplified samples from diploidy and triploidy samples. As shown in prior work, the log-scale CNV data for EGFR status can also exhibit heavily skewed distributions across a population of biopsy samples, which can manifest as a long tail with extremely large values (up to 22-fold log scale increase) in a relative minority of EGFR amplified samples. Such skewed distributions can present challenges for ML training, which can be addressed using data transformation. For instance, transformation can be used to maintain identical biopsy sample ordering between transformed and original scales, but to condense the spacing between samples with extreme values on the transformed scale, such that the distribution width of samples with CNV>3.5 approximated that of the samples with CNV<3.5.

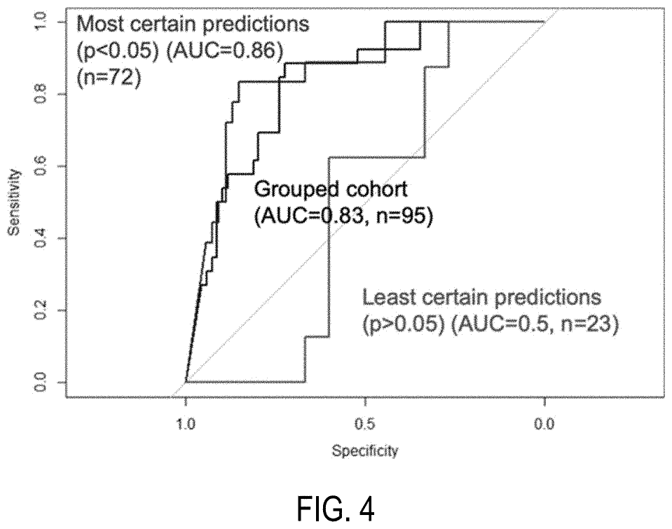

A dataset of 95 image-localized biopsies from a cohort of 25 primary GBM patients was collected. The EGFR amplification status for each biopsy sample was quantified and compared with spatially matched image features from corresponding multi-parametric MRI, which included conventional and advanced (diffusion, diffusion tensor, perfusion) MRI techniques. These spatially matched datasets were used to construct a transductive learning GP model to classify EGFR amplification status. This model quantified predictive uncertainty for each sample by measuring predictive variance and the distribution mean for each predicted sample, relative to the training model domain. These features were used to test the hypothesis that the sample belongs to the class predicted by the distribution mean (H1) versus not (H0), based on a standard one-sided z-test. This generated a p-value for each sample, which reflected the uncertainty of the prediction, such that smaller p-values corresponded with lower predictive uncertainty (i.e., higher certainty). These uncertainty estimates were integrated using transductive learning to optimize model training and predictive performance. In leave-one-patient-out cross validation, the model achieved an overall accuracy of 75% (77% sensitivity, 74% specificity) across the entire pooled cohort (n=95), without stratifying based on the certainty of the predictions. FIG. 4 illustrates how the spatially resolved predictive maps correspond with stereotactic biopsies from the regionally distinct genetic subpopulations that can co-exist within a single patient's GBM tumor.

For data-driven approaches like ML, predictions can be either interpolated between known observations (i.e., training data) within the model domain, or extrapolated by extending model trends beyond the known observations. Generally speaking, extrapolation carries greater risk of uncertainty, while interpolation is considered more reliable. It is contemplated that prioritizing interpolation (over extrapolation) during model training can reduce the uncertainty of radiogenomics predictions while improving overall model performance.

As a baseline representation of current methodology, a standard GP model was trained without the transductive learning capability to prioritize maximal predictive accuracy for distinguishing EGFR amplification status, irrespective of the type of prediction (e.g., interpolation versus extrapolation). This standard model was applied to quantify which sample predictions in the cohort (n=95) were interpolated (p<0.05) versus extrapolated (p>0.05, standard deviation >0.4).

When comparing with the standard GP model as the representative baseline, the transductive learning GP model shifted the type of prediction from extrapolation to interpolation in 11.6% (11/95) of biopsy cases. Amongst these sample predictions, classification accuracy increased from 63.6% with extrapolation (7/11 correctly classified) to 100% with interpolation (11/11 correctly classified). The transductive learning GP model also reduced the number of sample predictions that suffered from uncertainty due to inherent imperfections of model classification. This applied to those predictions with distributions that fell in close proximity to the classification threshold (so called border uncertainty), which associated with large p-values (p>0.05) and low standard deviation (<0.40). When comparing with the standard GP model as the representative baseline, the transductive learning GP model shifted the type of prediction, from border uncertainty to interpolation, in 15.8% (15/95) of biopsy cases. Amongst these sample predictions, classification accuracy dramatically increased from 40% in the setting of border uncertainty (6/15 correctly classified) to 93.3% with interpolation (14/15 correctly classified).

Substantially different sizes in feature sets and overall model complexity were observed when comparing the standard GP and transductive GP models. While the standard GP model selected 17 image features (across 5 different MRI contrasts), the transductive GP model selected only 4 features (across 2 MRI contrasts). The lower complexity of the transductive GP model likely stemmed from a key difference in model training: the transductive learning GP model first prioritized feature selection that minimized average predictive uncertainty (i.e., lowest sum of p-values), which helped to narrow candidate features to a relevant and focused subset. Only then did the model prioritize predictive accuracy within this smaller feature subset. Meanwhile, the standard GP model selected from the entire original feature set to maximize predictive accuracy, without constraints on predictive uncertainty. Although the accuracy was optimized on training data, the standard GP model could not achieve the same level of cross validated model performance (60% accuracy, 31% sensitivity, 71% specificity) compared to the transductive learning GP model, due largely to lack of control of extrapolation risks.

Existing published studies have used predictive accuracy to report model performance, but have not yet addressed model uncertainty. It is contemplated that leveraging both accuracy and uncertainty can further optimize model performance and applicability. When stratifying transductive learning GP sample predictions based on predictive uncertainty, a striking difference in model performance was observed. The subgroup of sample predictions with the lowest uncertainty (i.e., the most certain predictions) (p<0.05) (n=72) achieved the highest predictive performance (83% accuracy, 83% sensitivity, 83% specificity) compared to the entire cohort as a whole (75% accuracy, n=95). This could be explained by the substantially lower performance (48% accuracy, 63% sensitivity, 40% specificity) observed amongst the subgroup of highly uncertain sample predictions (p>0.05) (n=23).

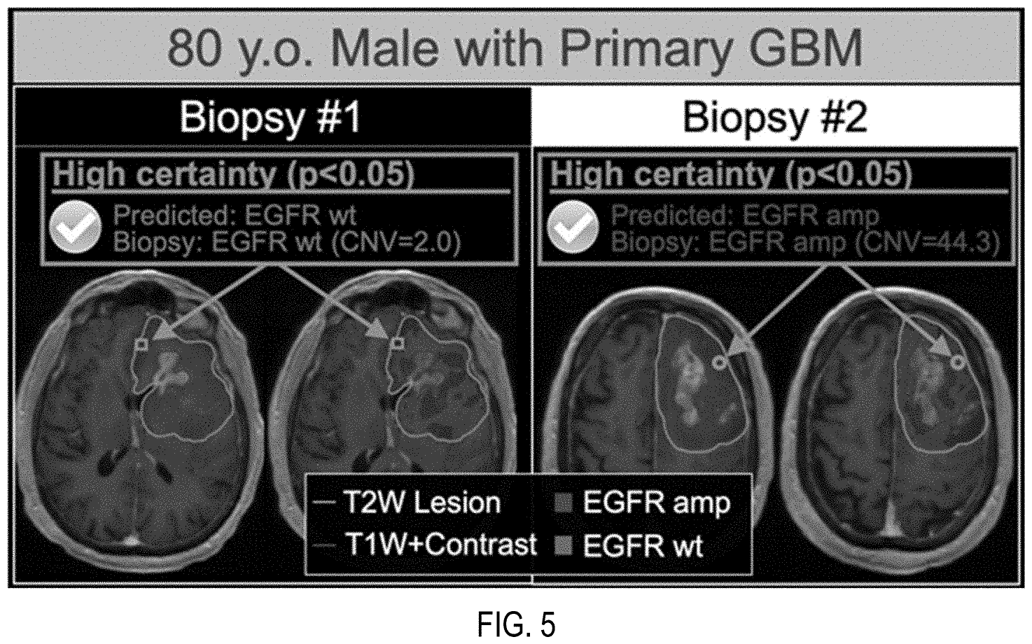

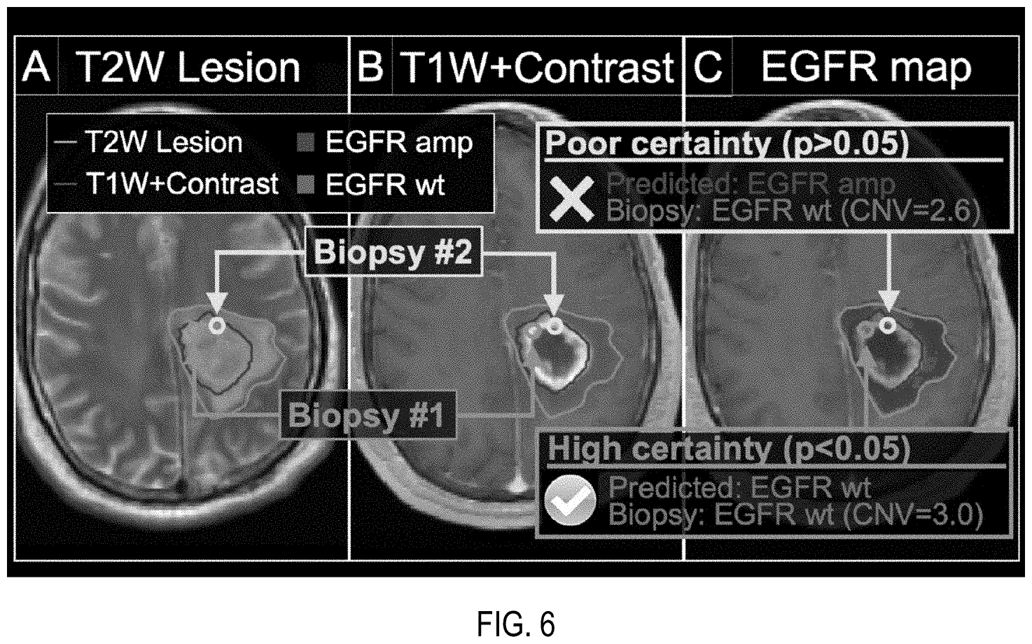

FIG. 5 shows the differences in model performance on ROC analysis when comparing sample predictions based on uncertainty. These discrepancies in model performance persisted even when stratifying with less stringent uncertainty thresholds (e.g., p<0.10, p<0.15). Together, these results suggest that predictive uncertainty can inform the likelihood of achieving an accurate sample prediction, which can help discriminate radiogenomics outputs, not only across patients, but at the regional level within the same patient tumor (FIG. 6).

In another example study, genetic prediction and/or other biological marker data associated with GBM were generated using a KGL model as described in the present disclosure.

GBM is the most aggressive type of brain tumor with median survival of 15 months. Intra-tumor molecular heterogeneity has been found to be one of the leading causes of treatment failure. Tumor cell density (“TCD”) is an important molecular marker to inform surgical intervention and radiation therapy. TCD is the percentage of tumor cells within a spatial unit of the tumor. TCD is spatially heterogeneous, meaning that TCD varies significantly across different sub-regions of each tumor. Mapping out the spatial distribution of TCD across each tumor is important for a neurosurgeon to determine where to resect. The mapping can also help radiation treatment planning by informing a radiation oncologist on how to optimize the spatial dose distribution according to the regional TCD. By providing a clinician with the additional information that can be generated using the systems and methods described in the present disclosure, overtreatment in some areas of the brain can be avoided, where such overtreatment would otherwise cause functional impairment, undertreatment of other areas, and lead to tumor recurrence. To know the TCD at each sub-region of a tumor, biopsy is the gold-standard approach. However, due to its invasive nature, only a few biopsy samples can be taken. MRI portrays the entire brain non-invasively. But MRI does not directly measure TCD while only providing proxy data. In this experiment, the KGL model described in the present disclosure was used to predict regional TCD of each tumor by integrating MRI, biopsy samples, and mechanistic model/domain knowledge.

In the example study, data were acquired from 18 GBM patients. Each patient had 2-14 biopsy samples, making a total of 82 samples. Pre-operative MRI including T1-weighted contrast-enhanced (T1+C) and T2-weighted sequences (T2) were used to guide biopsy selection. Neurosurgeons recorded biopsy locations via screen capture to allow subsequent co-registration with multiparametric MRI. The TCD of each biopsy specimen was assessed by a neuropathologist.

Each patient went through an MRI exam prior to treatment. The MRI exam produced multiple contrast images such as T1+C, T2, dynamic contrast enhancement (EPI+C), mean diffusivity (MD), fractional anisotropy (FA), and relative cerebral blood volume (rCBV). To extract features, an 8×8 pixel window was placed at each pixel as the center within a pre-segmented tumoral region-of-interest (ROI), which is an abnormality visible on T2. The window was slid throughout the entire ROI, and at each pixel, the average gray-level intensity was computed within the 8×8 pixel window from each of the six contrast images and used as features. Therefore, six image features were included in model training.

Biopsy samples are labeled samples as they have TCD. Samples corresponding to the sliding windows, except the windows at biopsy locations, are unlabeled as they only have image features not TCD.

A proliferation-invasion (PI) mechanistic model is integrated into the KGL model. PI is a PDE-based simulator driven by biological knowledge of how GBM tumor cells proliferate and invade to sounding brain tissues. The PDE for the PI model is:

$$\overbrace{\frac{\partial c}{\partial t}}^{Rate of Change \\ of Cell Density} = \overbrace{∇ \cdot ( D ( x ) ∇ c )}^{Invasion of Cells \\ into Nearby Tissue} + \overbrace{ρ c ( 1 - \frac{c}{K} )}^{Proliferation \\ of Cells}$$

where c(x, t) is the TCD at location x of the brain and time t, D(x) is the net rate of diffusion, ρ is the net rate of proliferation and K is the cell carrying capacity. The input to the PI model is the T1+C and T2 images of a patient, which are used to calibrate the model parameters. The output of PI includes the TCD estimate at each pixel. The PI map can capture some general trend of the spatial TCD distribution, but may lack localized precision due to simplified assumptions. The PI simulator was run for each patient to generate a PI map to be integrated with the KGL model.

In KGL, domain knowledge is incorporated through imposing constraints on the predictive means of unlabeled samples, i.e., g(E(ƒ_{L+1}), . . . , E(ƒ_{L+U}))≤ξ. Due to the aforementioned properties of the PI map, the PI maps can be used to regularize the general spatial trend of the TCD predictions. For example, based on the pixel-wise estimates of TCD generated by PI, the average estimate over 64 pixels within each 8×8 pixel window corresponding to an unlabeled sample can be computed. Denoting this average estimate for each unlabeled sample i by PIᵢ, i=L+1, . . . , L+U, the proposed constraints can be written as,

$$\begin{matrix} \{ \begin{matrix}{g}_{1} ( E ( {f}_{L + 1} ) , . . . , E ( {f}_{L + U} ) ) {=}^{Δ} \vert E ( {f}_{L + 1} ) - P {I}_{L + 1} \vert \leq {ξ}_{1} \\ ⋮ \\ {g}_{U} ( E ( {f}_{L + 1} ) , . . . , E ( {f}_{L + U} ) ) {=}^{Δ} \vert E ( {f}_{L + U} ) - P {I}_{L + U} \vert \leq {ξ}_{U}\end{matrix} , & ( 27 ) ; \\ {g}_{U + 1} ( E ( {f}_{L + 1} ) , . . . , E ( {f}_{L + U} ) ) {=}^{Δ} {Σ}_{i = L + 1 , . . . , L + U ; j > i} {{w}_{i j} ( E ( {f}_{i} ) - E ( {f}_{j} ) )}^{2} \leq {ξ}_{U + 1} , & ( 28 ) ; \\ {ξ}_{1} , . . . , {ξ}_{U + 1} \geq 0 , {Σ}_{i = 1}^{U + 1} {ξ}_{i} \leq ∈ , & ( 29 ) ;\end{matrix}$$

where wᵢⱼ=e^{-(PI}^{ᵢ}^{-PI}^{ⱼ}⁾^{²}. The constraints in Eqn. (27) encourage similarity between the predictive mean and the PI estimate at the same location (unbiopsied sample). Additionally, the constraint in Eqn. (28) encourages the predictive means of two samples to be similar if their PI estimates are similar, where the PI similarity is reflected by wᵢⱼ. Furthermore, considering that the PI map only provides approximates of the TCDs, a slack variable approach is used in (29) to make these constraints soft instead of hard constraints.

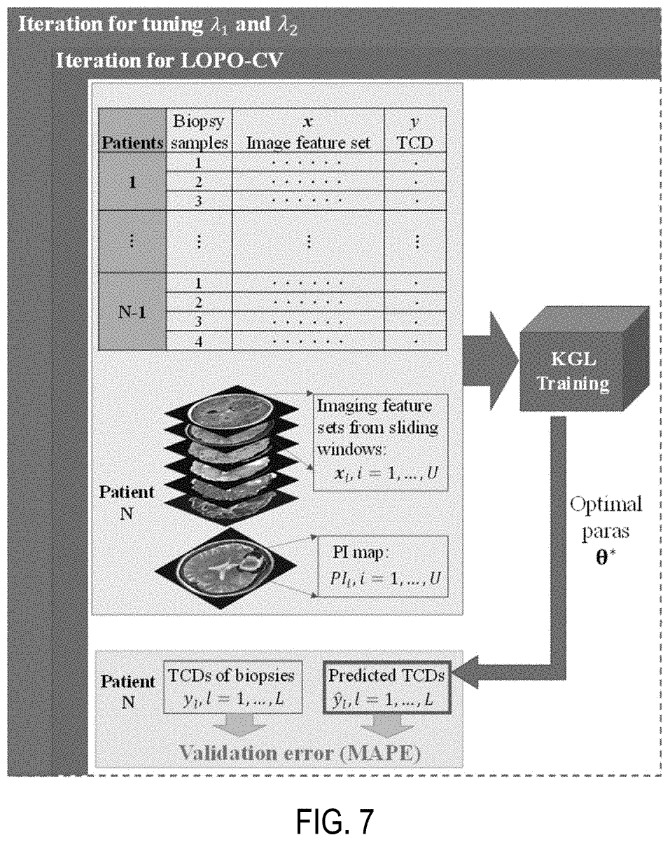

Model training is performed to determine the optimal parameter estimates, θ*, of the KGL model and to select the tuning parameters, λ₁ and λ₂. An example of a training procedure that can be used, and which was used in this example study, is shown in FIG. 7. The search for the optimal turning parameters is used as the outermost iteration. At fixed λ₁ and λ₂, the KGL optimization is solved for each patient. The input to the patient-specific optimization includes labeled samples from other patients, unlabeled samples from the particular patient, and the PI map of the particular patient. To improve efficiency and robustness, a subset of the first 100 unlabeled samples with the smallest average distances from the labeled samples was included in this example. The output of the model training is optimal parameters, θ*(λ₁, λ₂).

The KGL model under the optimal parameters is then used to generate a predictive distribution of the TCD for each biopsy sample of the particular patient. The predictive means of all the biopsy samples were compared with the true TCDs to compute the mean absolute prediction error (“MAPE”). This process was iterated with every patient in the dataset treated as “the particular patient”, known as leave-one-patient-out cross validation (LOPO-CV). While other types of CV schemes may be adopted, LOPO-CV aligned well with the natural grouping of samples in the example dataset. Finally, the best tuning parameters λ₁* and λ₂* were selected as the ones minimizing the average MAPE over all the patients. Under the λ₁* and λ₂*, the KGL optimization is solved for each patient to generate the final optimal parameters θ*for the patient.

The trained model can be used to generate a predictive distribution of the TCD for each sample (i.e., each sliding window) within the ROI. The predictive means of all the samples can be visualized by a color map overlaid on the ROI. Also, the predictive variances can be used to quantify prediction uncertainty, as described above.

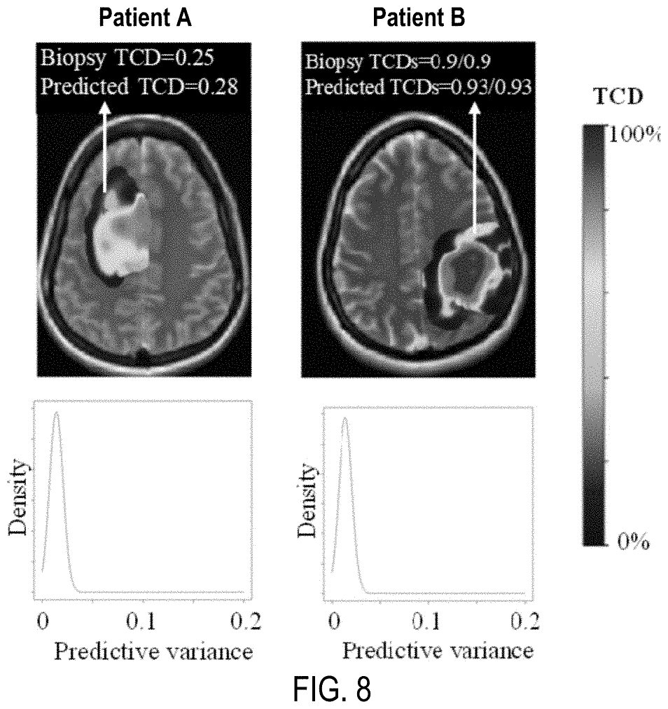

FIG. 8 shows the predictive TCD maps from two patients as examples. Colors represent the predictive means of the TCD from 0% (darkest blue) to 100% (darkest red). Below each map, the distribution of the predictive variances for samples within the ROI are also shown. Patient A had one biopsy sample shown on this slice of the MRI, and Patient B had two biopsy samples. The color maps produced by KGL show improved spatial smoothness and aligned better with the expected tumor cell distributions from known biology, especially for the color map of patient B. This is a benefit due to incorporation of the PI map/domain knowledge in model training. Furthermore, the predictive variance distribution by KGL is much more concentrated at the low variance range.

With a predicted TCD map for each patient, a neurosurgeon can have a better reference to decide which regions of the brain should have more (or less) cancerous tissues removed. For example, areas with high TCD can be maximally resected, whereas areas with little TCD could be preserved so as to protect the integrity of brain functions. This level of spatial precision is highly valuable for optimizing the surgical outcomes of GBM. Furthermore, the predicted TCD maps can also help radiation oncologists decide how to optimize the spatial radiation dose in radiation therapy. Areas with higher TCD should be irradiated more to kill the cancer cells, whereas areas with lower TCD should receive less dose to minimize radiation-induced complications. This level of spatial precision is much desirable for radiation treatment planning optimization. Finally, because KGL also generates a predictive variance in addition to the mean for each sample, the variance can be used to quantify the uncertainty of the prediction to guide more informed and risk-conscious clinical decision making.

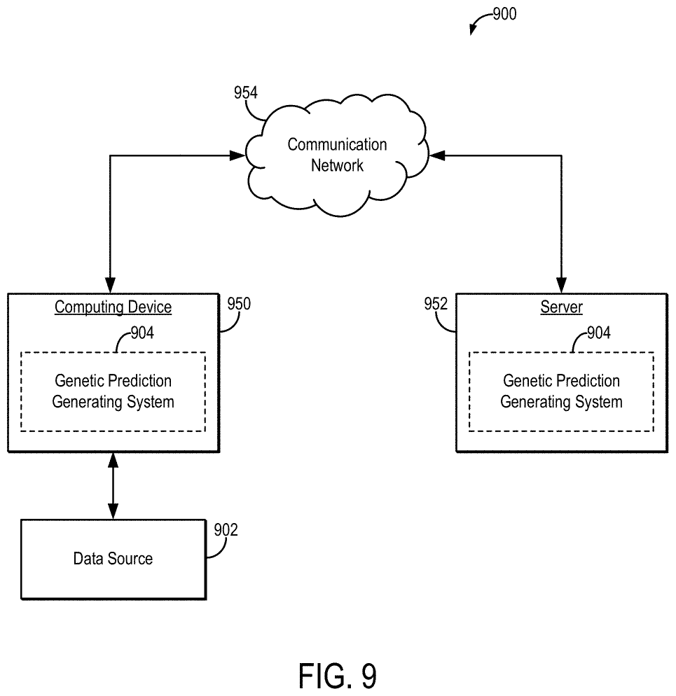

Referring now to FIG. 9, an example of a system 900 for generating genetic prediction data, or other biological prediction data and corresponding predictive uncertainty data in accordance with some embodiments of the systems and methods described in the present disclosure is shown. As shown in FIG. 9, a computing device 950 can receive one or more types of data (e.g., medical image data, training data) from data source 902, which may be a medical image data source. In some embodiments, computing device 950 can execute at least a portion of a genetic prediction generating system 904 to generate genetic prediction data, or other biological prediction data and corresponding predictive uncertainty data from data received from the data source 902.

Additionally or alternatively, in some embodiments, the computing device 950 can communicate information about data received from the data source 902 to a server 952 over a communication network 954, which can execute at least a portion of the genetic prediction generating system. In such embodiments, the server 952 can return information to the computing device 950 (and/or any other suitable computing device) indicative of an output of the genetic prediction generating system.

In some embodiments, computing device 950 and/or server 952 can be any suitable computing device or combination of devices, such as a desktop computer, a laptop computer, a smartphone, a tablet computer, a wearable computer, a server computer, a virtual machine being executed by a physical computing device, and so on. The computing device 950 and/or server 952 can also reconstruct images from the data.

In some embodiments, data source 902 can be any suitable source of image data (e.g., measurement data, images reconstructed from measurement data), such as an MRI system, another computing device (e.g., a server storing image data), and so on. In some embodiments, data source 902 can be local to computing device 950. For example, data source 902 can be incorporated with computing device 950 (e.g., computing device 950 can be configured as part of a device for capturing, scanning, and/or storing images). As another example, data source 902 can be connected to computing device 950 by a cable, a direct wireless link, and so on. Additionally or alternatively, in some embodiments, data source 902 can be located locally and/or remotely from computing device 950, and can communicate data to computing device 950 (and/or server 952) via a communication network (e.g., communication network 954).

In some embodiments, communication network 954 can be any suitable communication network or combination of communication networks. For example, communication network 954 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 108 can be a local area network, a wide area network, a public network (e.g., the Internet), a private or semi-private network (e.g., a corporate or university intranet), any other suitable type of network, or any suitable combination of networks. Communications links shown in FIG. 9 can each be any suitable communications link or combination of communications links, such as wired links, fiber optic links, Wi-Fi links, Bluetooth links, cellular links, and so on.

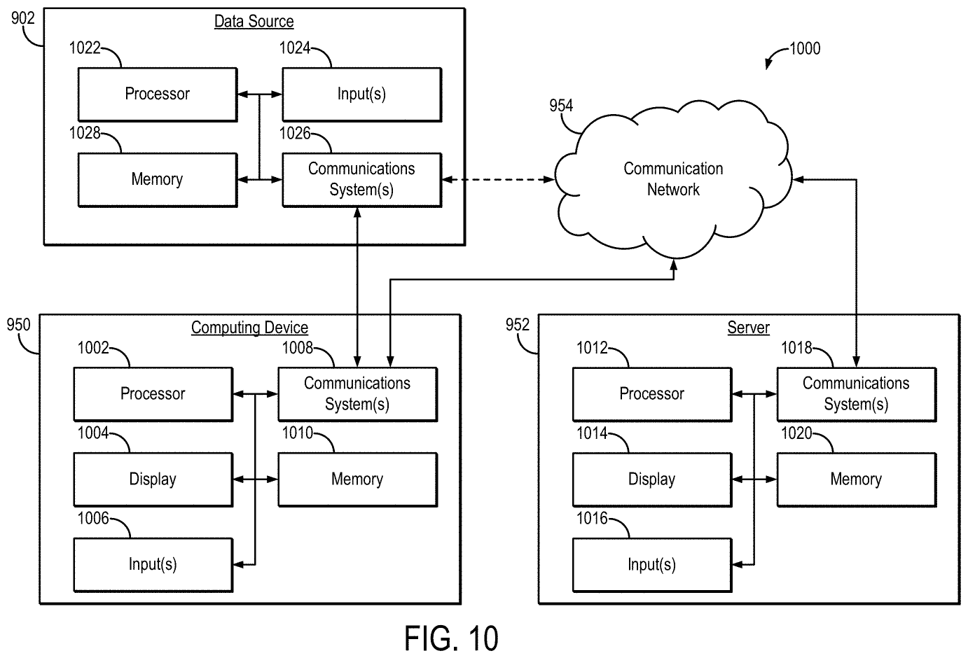

Referring now to FIG. 10, an example of hardware 1000 that can be used to implement data source 902, computing device 950, and server 954 in accordance with some embodiments of the systems and methods described in the present disclosure is shown. As shown in FIG. 10, in some embodiments, computing device 950 can include a processor 1002, a display 1004, one or more inputs 1006, one or more communication systems 1008, and/or memory 1010. In some embodiments, processor 1002 can be any suitable hardware processor or combination of processors, such as a central processing unit (“CPU”), a graphics processing unit (“GPU”), and so on. In some embodiments, display 1004 can include any suitable display devices, such as a computer monitor, a touchscreen, a television, and so on. In some embodiments, inputs 1006 can include any suitable input devices and/or sensors that can be used to receive user input, such as a keyboard, a mouse, a touchscreen, a microphone, and so on.

In some embodiments, communications systems 1008 can include any suitable hardware, firmware, and/or software for communicating information over communication network 954 and/or any other suitable communication networks. For example, communications systems 1008 can include one or more transceivers, one or more communication chips and/or chip sets, and so on. In a more particular example, communications systems 1008 can include hardware, firmware and/or software that can be used to establish a Wi-Fi connection, a Bluetooth connection, a cellular connection, an Ethernet connection, and so on.

In some embodiments, memory 1010 can include any suitable storage device or devices that can be used to store instructions, values, data, or the like, that can be used, for example, by processor 1002 to present content using display 1004, to communicate with server 952 via communications system(s) 1008, and so on. Memory 1010 can include any suitable volatile memory, non-volatile memory, storage, or any suitable combination thereof. For example, memory 1010 can include RAM, ROM, EEPROM, one or more flash drives, one or more hard disks, one or more solid state drives, one or more optical drives, and so on. In some embodiments, memory 1010 can have encoded thereon, or otherwise stored therein, a computer program for controlling operation of computing device 950. In such embodiments, processor 1002 can execute at least a portion of the computer program to present content (e.g., images, user interfaces, graphics, tables), receive content from server 952, transmit information to server 952, and so on.

In some embodiments, server 952 can include a processor 1012, a display 1014, one or more inputs 1016, one or more communications systems 1018, and/or memory 1020. In some embodiments, processor 1012 can be any suitable hardware processor or combination of processors, such as a CPU, a GPU, and so on. In some embodiments, display 1014 can include any suitable display devices, such as a computer monitor, a touchscreen, a television, and so on. In some embodiments, inputs 1016 can include any suitable input devices and/or sensors that can be used to receive user input, such as a keyboard, a mouse, a touchscreen, a microphone, and so on.

In some embodiments, communications systems 1018 can include any suitable hardware, firmware, and/or software for communicating information over communication network 954 and/or any other suitable communication networks. For example, communications systems 1018 can include one or more transceivers, one or more communication chips and/or chip sets, and so on. In a more particular example, communications systems 1018 can include hardware, firmware and/or software that can be used to establish a Wi-Fi connection, a Bluetooth connection, a cellular connection, an Ethernet connection, and so on.

In some embodiments, memory 1020 can include any suitable storage device or devices that can be used to store instructions, values, data, or the like, that can be used, for example, by processor 1012 to present content using display 1014, to communicate with one or more computing devices 950, and so on. Memory 1020 can include any suitable volatile memory, non-volatile memory, storage, or any suitable combination thereof. For example, memory 1020 can include RAM, ROM, EEPROM, one or more flash drives, one or more hard disks, one or more solid state drives, one or more optical drives, and so on. In some embodiments, memory 1020 can have encoded thereon a server program for controlling operation of server 952. In such embodiments, processor 1012 can execute at least a portion of the server program to transmit information and/or content (e.g., data, images, a user interface) to one or more computing devices 950, receive information and/or content from one or more computing devices 950, receive instructions from one or more devices (e.g., a personal computer, a laptop computer, a tablet computer, a smartphone), and so on.

In some embodiments, data source 902 can include a processor 1022, one or more inputs 1024, one or more communications systems 1026, and/or memory 1028. In some embodiments, processor 1022 can be any suitable hardware processor or combination of processors, such as a CPU, a GPU, and so on. In some embodiments, the one or more inputs 1024 are generally configured to acquire data, images, or both, and can include an MRI system. Additionally or alternatively, in some embodiments, the input(s) 1024 can include any suitable hardware, firmware, and/or software for coupling to and/or controlling operations of an MRI system. In some embodiments, one or more portions of the input(s) 1024 can be removable and/or replaceable.

Note that, although not shown, data source 902 can include any suitable inputs and/or outputs. For example, data source 902 can include input devices and/or sensors that can be used to receive user input, such as a keyboard, a mouse, a touchscreen, a microphone, a trackpad, a trackball, and so on. As another example, data source 902 can include any suitable display devices, such as a computer monitor, a touchscreen, a television, etc., one or more speakers, and so on.

In some embodiments, communications systems 1026 can include any suitable hardware, firmware, and/or software for communicating information to computing device 950 (and, in some embodiments, over communication network 954 and/or any other suitable communication networks). For example, communications systems 1026 can include one or more transceivers, one or more communication chips and/or chip sets, and so on. In a more particular example, communications systems 1026 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 1028 can include any suitable storage device or devices that can be used to store instructions, values, data, or the like, that can be used, for example, by processor 1022 to control the one or more image acquisition systems 1024, and/or receive data from the one or more image acquisition systems 1024; to images from data; present content (e.g., images, a user interface) using a display; communicate with one or more computing devices 950; and so on. Memory 1028 can include any suitable volatile memory, non-volatile memory, storage, or any suitable combination thereof. For example, memory 1028 can include RAM, ROM, EEPROM, one or more flash drives, one or more hard disks, one or more solid state drives, one or more optical drives, and so on. In some embodiments, memory 1028 can have encoded thereon, or otherwise stored therein, a program for controlling operation of data source 902. In such embodiments, processor 1022 can execute at least a portion of the program to generate images, transmit information and/or content (e.g., data, images) to one or more computing devices 950, receive information and/or content from one or more computing devices 950, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A method for generating biological marker prediction data from medical image data, the method comprising:
(a) accessing a trained machine learning model with a computer system, wherein the machine learning model has been trained in order to generate biological marker prediction data and corresponding predictive uncertainty data from medical image data;
(b) accessing medical image data with the computer system, wherein the medical image data comprise medical images of a subject obtained using a medical imaging system;
(c) inputting the medical image data to the trained machine learning model, generating output as biological marker prediction data and corresponding predictive uncertainty data, wherein the biological marker prediction data comprise biological marker predictions and corresponding predictive uncertainty data comprise quantitative measures of an uncertainty of each biological marker prediction in the biological marker prediction data; and
(d) displaying the biological marker prediction data and predictive uncertainty data to a user.

2. The method of claim 1, wherein the machine learning model is a Gaussian process model.

3. The method of claim 2, wherein the Gaussian process model is a transductive learning Gaussian process model.

4. The method of claim 1, wherein the machine learning model is a knowledge-infused global-local data fusion model.

5. The method of claim 4, wherein the knowledge-infused global-local data fusion model is trained on training data comprising labeled biopsy samples as local data and medical imaging data as unlabeled global data.

6. The method of claim 4, wherein the knowledge-infused global-local data fusion model integrates output data generated by a mechanistic model.

7. The method of claim 6, wherein the mechanistic model is a proliferation-invasion model.

8. The method of claim 7, wherein the output data generated by the mechanistic model is a proliferation-invasion parameter map.

9. The method of claim 1, wherein the machine learning model is trained on training data comprising image-localized tissue biopsy samples.

10. The method of claim 9, wherein the machine learning model is trained on the training data using transductive learning, wherein the training data comprises both labeled samples and unlabeled samples.

11. The method of claim 10, wherein the machine learning model is trained on the training data using transductive learning by:
generating predictions for the unlabeled samples by applying the machine learning model to the labeled samples;
generating a combined data set that combines the predictions for the unlabeled samples with the training data; and
generating a predictive distribution for each new test sample using the combined data set.

12. The method of claim 1, wherein the medical image data comprise magnetic resonance image data.

13. The method of claim 12, wherein the magnetic resonance image data comprise multiparametric magnetic resonance image data containing magnetic resonance images representative of a plurality of different magnetic resonance image contrast types.

14. The method of claim 13, wherein the plurality of different magnetic resonance image contrast types comprise at least two of T1-weighting, T1-weighting with a contrast agent, T2-weighting, diffusion weighting, and perfusion weighting.

15. The method of claim 12, wherein the magnetic resonance image data comprise both magnetic resonance images and parametric maps representative of quantitative parameters generated using the magnetic resonance images.

16. The method of claim 15, wherein the parametric maps comprise at least one of T1 maps, T2 maps, apparent diffusion coefficient maps, mean diffusivity maps, fractional anisotropy maps, cerebral blood volume maps, cerebral blood flow maps, and mean transit time maps.

17. The method of claim 1, wherein the biological marker prediction data comprise genetic prediction data that indicate predictions of genetic features in the subject.

18. The method of claim 1, wherein the biological marker prediction data comprise tissue characteristic prediction data that indicate predictions of tissue characteristics in the subject.

19. The method of claim 18, wherein the tissue characteristics include at least one of molecular pathways, quantity of tumor cell density, location of tumor cell density, and non-tumoral cells type classification.
